(19) United States
(12) Patent Application Publication
(10) Pub. No.: US 2008/0249587 A1
(43) Pub. Date: Oct. 9, 2008

(54) MICROCURRENT STIMULUS APPARATUS

(75) Inventors: Sung Lae Cho, Busan (KR); Jin Book Moon, Busan (KR); Seong Jin Koo, Busan (KR); Yun Seok Jang, Busan (KR)

Correspondence Address: Hyun Jong Park;TUCHMAN & PARK LLC, 41 White Birch Road, Redding, CT, 06896-2209, US

(73) Assignee: G-Man Co., Ltd., Busan (KR)

(21) Appl. No.: 12/139,474

(22) Filed: Jun. 14, 2008

Related U.S. Application Data
(63) Continuation of application No. PCT/KR06/05445, filed on Dec. 14, 2006.

(30) Foreign Application Priority Data
Dec. 14, 2005 (KR) 20-2005-0035223
Oct. 31, 2006 (KR) 20-2006-0028780

(51) Int. Cl. A61N 1/34

(52) U.S. Cl. 607/46

(57) ABSTRACT

Disclosed therein are a coupling boss and a method for fabricating a coupling boss, that a collar part is formed through a collar drawing process using a metal plate material, and then, a body part of the coupling boss is firmly combined to a substrate plate through processes of deep drawing, tapping, cleansing, plating and iron coining in order, thereby maximizing competitive power in price, miniaturizing the coupling boss in size, and maximizing tensile strength, compression strength, and surface roughness. In this instance, a more stable material thickness can be kept by performing a collar drawing process or a curling process when a screw part of the coupling boss is formed. The processes of: forming a collar part (2) through a collar drawing process for forming a screw part (8) of the coupling boss (1) using a metal plate material; forming a body part (3) and a flange part (4) on the metal plate material, on which the collar part (2) is formed, through a deep drawing process; and tapping to form the screw part (8) on the inner peripheral portion of the collar part (2) and the processes of: forming a body part (3) and a flange part (4) by a deep drawing process using a metal plate material; forming a collar part (2) on the body part (3) by a curling process; and tapping to form a screw part (8) on the inner peripheral portion of the collar part (2) are performed selectively. After that, the formed coupling boss (1) is firmly combined to a substrate plate (5) through the processes of cleansing, plating, and iron coining.

### REFERENCE TO RELATED APPLICATIONS

This a continuation of pending International Patent Application PCT/KR2006/005445 filed on Dec. 14, 2006, which designates the United States and claims priorities of Korean Patent Application No. 20-2005-0035223 filed on Dec. 14, 2005; Korean Patent Application No. 20-2006-0028780 filed on Oct. 31, 2006.

### FIELD OF THE INVENTION

The present invention relates to a microcurrent stimulus apparatus. More particularly, the present invention relates to a microcurrent stimulus apparatus which supplies microcurrent to a user's body part so that a user receives a massage at his or her body part through a stimulus plate by wearing the microcurrent stimulus apparatus around his or her ankle or neck.

### BACKGROUND OF THE INVENTION

Recently, people are becoming more interested in their health, so a variety of health-care apparatuses has been developed and used for curing pains and aches in people's body parts.

Particularly, a microcurrent stimulus apparatus, which stimulates meridian loci of body parts using microcurrent generated from a microcurrent generation unit, is generally used in order to cure obesity and pains.

From the viewpoint of oriental medicine, a foot of a human is considered as important a body part as the heart and is called a secondary heart because it has apexes connected to a variety of organs of the body via nerves, a great deal of capillary vessels, autonomic nerves, the Achilles tendon, and one or more sphygmuses which are disposed at the back thereof.

The heart sends blood throughout the body by self-contraction and self-expansion movements. Unlike the heart, which contracts and expands by itself, the feet should be externally stimulated through working or running in order to receive an acupressure effect at acupuncture loci thereof, so that organs in body parts connected to the acupuncture loci via the nerves are affected.

A variety of apparatuses which directly or indirectly cures body parts using acupuncture loci distributed on the foot are widely used. Suji Chim, which is a treatment for applying acupuncture to the fingers, and Chock Chim, which is a treatment for applying acupuncture to the feet, are representative examples. Suji Chim and Chock Chim are used to stimulate acupuncture loci distributed on the hands and feet and connected to particular organs of the body via nerves.

For other external stimulation methods and apparatuses for treating body parts, in addition to Chim treatments, a variety of physical apparatuses and methods, such as acupressure therapy, cautery, low frequency therapy, magnetic field therapy, and electric stimulation therapy is frequently used. Further, as it is known that microcurrent of several to several hundreds of microamperes is useful to stimulate acupuncture loci, a variety of microcurrent stimulus apparatuses using microcurrent have been developed and are widely used.

However, most of the conventional microcurrent stimulus apparatuses are expensive, and thus most people cannot easily use such apparatuses. Further, since such apparatuses are heavy and have a large size, they are not portable, thus people can use them only at the location where they are installed, such as one's home or office.

In order to solve such problems, shoes, in which microcurrent flows due to power generated when a force such as impact or pressure is applied to a piezoelectric element, have been disclosed. However, in the case of using such piezoelectric elements, it is impossible to control the flow of microcurrent as desired by a user, even if a user wants to stop the flow of microcurrent. For example, if a user wears shoes having the piezoelectric elements therein on rainy days or while playing sports, the user may not want microcurrent to flow through the shoes. However, the user cannot stop the flow of microcurrent as long as the user is walking or running.

On the other hand, among housekeepers and people who use computers for long periods, the number of people experiencing pains or aches in their shoulders is increasing. Particularly, when people care a lot about their work, are absorbed in their jobs or get stressed, blood flow is slowed, so chronic fatigue and stress syndromes such as stiffness and heaviness of the neck and shoulders, impaired memory and concentration, amblyopia, insomnia, and fatigue result. In oriental medicine, severe pain is called “Hyunbyuk.” Pains and aches in the shoulders are a symptom of Hyunbyuk. This is generally the result of a lack of vital energy. Accordingly, in order to treat these symptoms, oriental medicines (herbs) or western medicines have generally been used.

However, in the case of taking herbs or medicines, there is a problem that immune deficiency can result. Accordingly, a variety of health-care apparatuses for curing and preventing sickness without using medicines have been developed and are widely used. Among them, oriental treatments such as acupuncture and moxa cautery, which are essential treatments for facilitating vital energy flow and blood circulation, are considered important treatments.

In addition to the acupuncture and cautery treatments, acupressure and massage treatments, which stimulate meridian loci and meridian muscles, have recently received a lot of attention. However, the acupuncture and cautery treatments have a problem in that it is difficult for unskilled people to perform such treatments and only experts in the stimulation of meridian muscles and spots suitable for acupuncture can properly perform the treatments.

Accordingly, needed is an apparatus which can be easily manipulated by even unskilled people as well as experts and is capable of facilitating blood flow and vital energy circulation when it is applied to acupuncture loci, in order to ease stiffness and heaviness of the muscles of the shoulders, neck and back, caused by accumulated stress, and pains in the waist and to normalize blood and vital energy circulation throughout the body.

### SUMMARY OF THE INVENTION

Accordingly, the present invention has been devised in consideration of the aforementioned problems and conditions, and it is an object of the present invention to provide a microcurrent stimulus apparatus which can give helpful stimulation to acupuncture loci or muscles of body parts having pains, such as the sole of the foot, shoulders, neck, back, and waist with microcurrent so that the stimulations can be transferred to organs of the human body through nerves, and has an advantageous effect that it is portable, so that a user can wear it around his or her neck or ankle and attaches a detachable patch-type stimulus plate to his or her neck or ankle.

It is a further object of the present invention to provide a microcurrent stimulus apparatus having a switch which can be turned on and off by a user so that it allows a user to control the on/off operation thereof.

It is a still further object of the present invention to provide a microcurrent stimulus apparatus having an indicator lamp for indicating a remaining battery level.

In order to achieve the above described advantageous effects and objects, in accordance with the present invention, there is provided a microcurrent stimulus apparatus comprising a main body part including a battery disposed in a casing for supplying power, a microcurrent generation unit for converting the power from the battery to microcurrent, a switch disposed on an outer surface of the casing for allowing the flow of the power from the battery to be controlled, and an indicator lamp for indicating remaining battery capacity; a stimulus plate including one or more acupuncture pressure patches connected to the main body part and exposed outside the casing by protruding from the outer surface of the casing, and acupuncture protrusions provided to the center portion of the acupuncture pressure patch for stimulating acupuncture loci and muscles of body parts with microcurrent; and a coupling part coupled to the main body part for allowing a user to carry the main body part by wearing the coupling part around or on his or her body parts.

The microcurrent generation unit comprises a condenser C, resistors R1-R2, a transistor Tr and a variable resistor VR, which are electrically connected to each other and generate microcurrent in a manner such that current caused by the power of the battery flows toward the variable resistor VR in order to charge the condenser C when the switch is turned on and is simultaneously supplied to a collector of the transistor Tr; the current flowing to the variable resistor VR until the condenser C is charged up flows toward the base of the transistor Tr as much as the voltage drop which is caused across the resistor R1 and the resistor R2; and the condenser C serves as a decoupling condenser when charging of the condenser C is finished, and thus the current is applied to the base of the transistor Tr.

The microcurrent generation unit generates microcurrent using the internal resistance of the acupuncture pressure protrusions.

The main body part may have a cover for allowing the battery to be replaced, and a flashing diode for indicating the supply of battery power, so that the flashing diode flashes at regular intervals when the switch is in an on-state, and the stitch may continuously remain in the on-state while the microcurrent is generated.

The acupuncture pressure protrusions of the stimulus plate may be made of magnets.

At least one of the microcurrent stimulus plates may be installed on an inner surface of the coupling part, so that it can stimulate acupuncture loci disposed at a user's ankle with microcurrent.

The coupling part may have a length adjustment unit for adjusting the length thereof, and the length adjustment unit may be a Velcro tape type, a buckle type, or a ring type.

The coupling part may be combined with the main body part so that a user can carry the main body part by wearing the coupling part around his or her neck.

As described above, the microcurrent stimulus apparatus according to the present invention has advantageous effects in that it is portable because it has a small size and volume, and in that it can stimulate acupuncture loci or muscles of the body parts, such as the ankle, neck, shoulders, back and waist, with microcurrent, so that such stimulation is transferred to organs of the body via nerves.

The microcurrent stimulus apparatus has further advantageous effects in that it allows a user to control the on/off operation thereof using a switch, and indicates remaining battery capacity, so a user can more effectively care for his or her health.

### BRIEF DESCRIPTION OF THE DRAWINGS

FIG. 1 is a diagram illustrating meridian pathways related to the organs of the human body;

FIG. 2 and FIG. 3 are diagrams illustrating acupuncture loci in the human body;

FIG. 4 is a diagram illustrating a microcurrent stimulus apparatus according the first embodiment of the present invention;

FIG. 5 is a perspective view illustrating the microcurrent stimulus apparatus according to the first embodiment of the present invention;

FIG. 6 is a perspective view illustrating the internal structure of the microcurrent stimulus apparatus according to the first embodiment of the present invention;

FIG. 7 is a circuit diagram illustrating a microcurrent generation unit of a microcurrent stimulus apparatus according to the embodiment of the present invention;

FIG. 8 is a circuit diagram illustrating a microcurrent generation unit of a microcurrent stimulus apparatus according to the second embodiment of the present invention;

FIG. 9 is a perspective view illustrating a microcurrent stimulus apparatus according to the third embodiment of the present invention;

FIG. 10 is a perspective view illustrating a microcurrent stimulus apparatus according to the fourth embodiment of the present invention;

FIG. 11 is a perspective view illustrating the internal structure of the microcurrent stimulus apparatus according to the fourth embodiment of the present invention;

FIG. 12 is a perspective view illustrating the state of the microcurrent stimulus apparatus according to the first embodiment of the present invention being worn around an ankle of a user; and

FIG. 13 is a perspective view illustrating the state of the microcurrent stimulus apparatus according to the fourth embodiment of the present invention being worn around a user's neck.

### DETAILED DESCRIPTION OF THE INVENTION

Hereinafter, a microcurrent stimulus apparatus according to embodiments of the present invention will be described in detail.

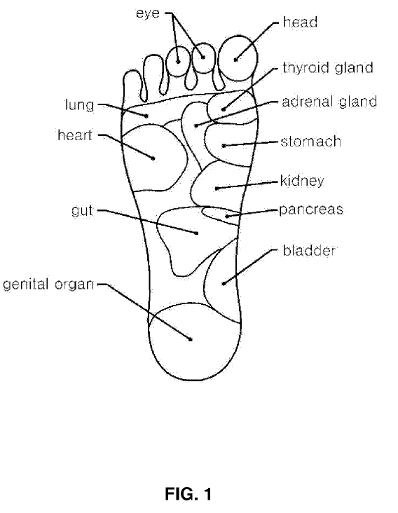

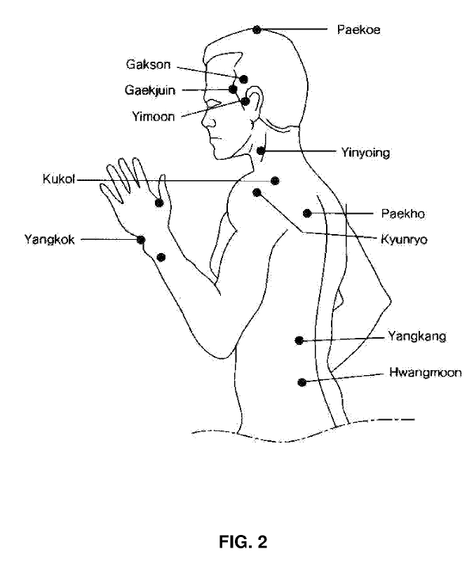

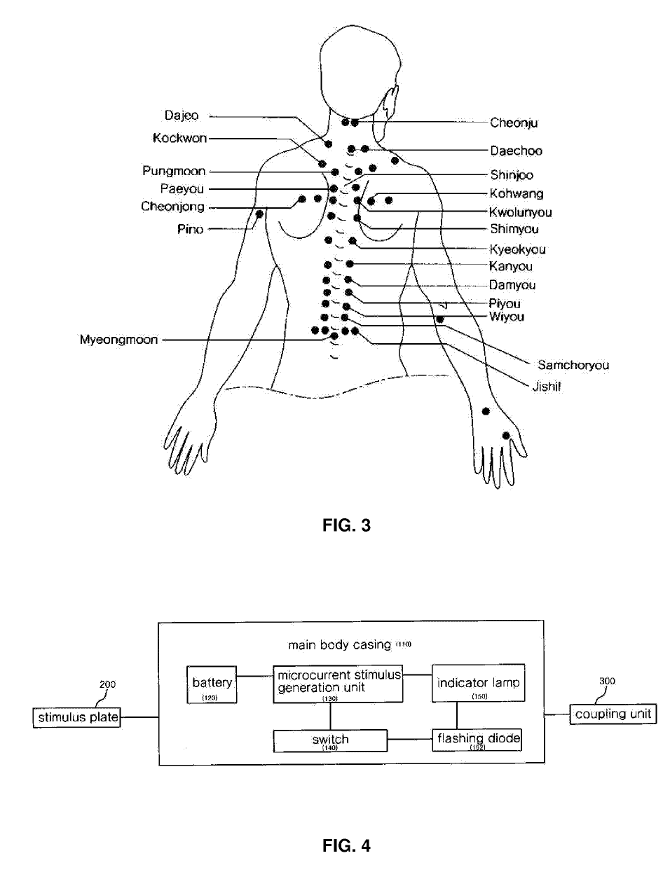

FIG. 1 illustrates nerve spots in the sole of a foot, which are connected to respective organs of the body, and FIG. 2 and FIG. 3 illustrate meridian pathways.

With reference to FIG. 1 through FIG. 3, the distribution of nerves in body parts will be described.

The distribution of nerves is not scientifically accepted by western medicine, but is well-known in oriental medicine. Particularly, the distribution of nerves is used in daily life, in practices such as Chock Chim therapy or foot massage therapy.

Nerve spots correlated with the organs of the body are distributed as shown in FIG. 1. For example, since diabetes mellitus is caused by an insulin secretion problem, for a diabetes mellitus patient, it is helpful to stimulate spots correlated with the pancreas, out of nerve spots (meridian loci). Further, for a patient having digestion problems, nerve spots correlated with the stomach should be stimulated.

As shown in FIG. 2, PaekoeHyul is disposed at the center of the top of the head, and is an acupuncture spot (acupoint) for curing headaches afflicting the entire head. KukolHyul is an acupoint disposed slightly above a spot where two bones (Kukol) cross each other and is for curing pains in shoulders. KyunryoHyul is a meridian locus used to treat obesity of the arms, and is disposed at a concave spot near the origin of the deltoid muscle.

As shown in FIG. 3, DajeoHyul, disposed between the first spinous process of the thoracic vertebrae and the second spinous process of the thoracic vertebrae, is known as a meridian locus for treating coughing in oriental medicine. ShinjuHyul is disposed at a spot in line with the lowest rib, and the spine is interposed between the two ShinjuHyuls. By stimulating the ShinjuHyuls, it is possible to relax the waist muscles and strengthen the function of the kidneys, so that pains in the waist are fundamentally cured.

The diagram showing the distribution of nerves is widely known in oriental medicine, so a detailed description thereof will be made herein.

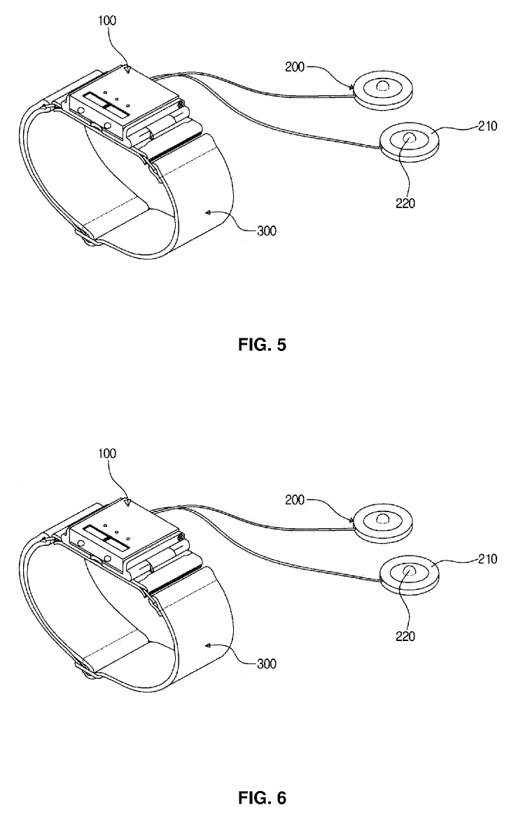

FIG. 4 is a block diagram illustrating the microcurrent stimulus apparatus according to the first embodiment of the present invention, FIG. 5 is a perspective view illustrating the microcurrent stimulus apparatus according to the first embodiment of the present invention, and FIG. 6 is a perspective view illustrating the internal structure of the microcurrent stimulus apparatus according to the first embodiment of the present invention.

Referring to FIG. 4 through FIG. 6, the microcurrent stimulus apparatus according to the first embodiment of the present invention includes a main body part 100 worn around a user's ankle using a coupling part 300, and a stimulus plate 200 electrically connected to the main body part 100 and disposed on the meridian loci on the sole of a foot for giving stimulation to the meridian loci with microcurrent, so that beneficial stimulation is transferred to the organs of the body.

The main body part 100 will be described in detail first.

The main body part 100 comprises a casing 110, a power source provided in the casing 100, a microcurrent generation unit 130 for converting power from the power source into microcurrent, a switch 140 for controlling the supply of power, and an indicator lamp 150 for indicating remaining battery capacity.

According to the embodiment of the present invention, the power source may be a battery 120.

As shown in FIG. 6, the battery 120 is installed in the casing 110 and supplies power to be used by the microcurrent generation unit 130 in order to generate microcurrent.

The battery 120 can be one of a variety of kinds. For example, the battery 120 can be a first battery, which is a disposable battery, such as a manganese battery or an alkaline battery, or it can be a secondary battery, which is a rechargeable battery, such as a nickel-cadmium (Ni—Cd) battery, a nickel-hydrogen (Ni-MH) battery, or a lithium-ion (Li-Ion) battery.

According to the embodiment of the present invention, the battery may be a general battery producing 1.5V, as shown in FIG. 6, but the battery according to present invention is not limited thereto. The battery 120 can be a rechargeable battery which is rechargeable using a supply voltage, or using a solar cell.

The casing 110 may preferably have a cover 112 so that the used battery 120 can be replaced.

The microcurrent generation unit 130 receives power from the power source and generates microcurrent to be used to stimulate meridian loci of the foot or meridian loci and muscles in body parts, such as the shoulders, neck, and waist. The microcurrent generation unit 130 includes a circuit board for generating microcurrent having a value in the range from several to several hundreds of microamperes, and is installed in the casing 110 together with the battery 120.

The microcurrent generation unit 130 is electrically connected to the stimulus plate 200 in contact with the sole of the foot via an electric wire, so that it is possible to supply microcurrent to the sole of the foot.

The microcurrent generation unit 130 is required to generate microcurrent having a value in the range from several to several hundreds of amperes, which is useful and suitable for electric stimulation. The reason follows. It is known that current of 0.06 milliamperes (mA) flows through the human body, and intensity of the current flowing through the body varies according to one's state of health. That is, it is known that less current than normal flows in unhealthy people. Accordingly, it is desirable to externally apply microcurrent, having intensity appropriate for stimulation of the human body, to a human body, because it contributes to internal balancing of the human body.

Further, it is known that 1 milliampere of current is sufficient to be sensed by a human being, although sensitivity varies according to individuals and a long period of current conduction through a human body is not desirable. Accordingly, a current of several to several hundreds of microamperes (μA), which is less then 1 milliampere (mA), when applied to body parts, such as the soles, neck, shoulders, back, or waist, will be sufficient. Preferably, the current may not be continuously applied but may be intermittently applied to body parts.

More preferably, microcurrent, having a strength such that it does not negatively affect the human body even when applied for a long time, and inflowing current less than the internal current of the body, that is, 300 microamperes, is applied to body parts.

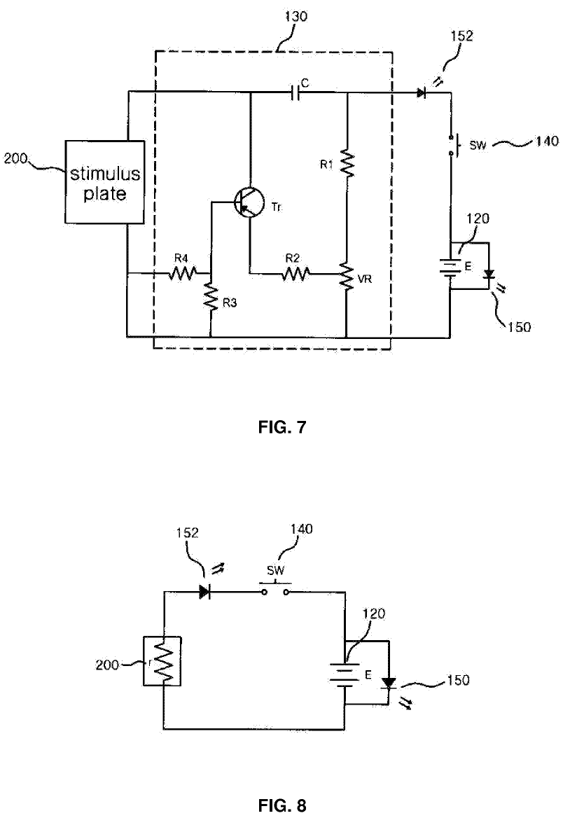

FIG. 7 is a circuit diagram illustrating the microcurrent generation unit 130 according to the first embodiment of the present invention.

As shown in FIG. 7, the microcurrent generation unit 130 comprises a condenser C, resistors R1 and R2, a transistor Tr and a variable resistor VR. The microcurrent generation unit 130 converts power E from the battery 120 to microcurrent in response to the on on/off manipulation of the switch 140, and thus the microcurrent is transmitted to the stimulus plate 200, which will be described below.

The battery 120 can be a battery pack comprising a plurality of normal batteries, each producing 1.5V, so that the total power of the battery 120 will be 3 to 12V. As the power varies from 3V to 12V, resistance of the variable resistor VR also varies, so that 1 through 300 microamperes of microcurrent can be generated.

The operation of the microcurrent generation unit 130 will be described below.

If the switch 140 is turned on, direct current supplied from the battery 120 flows toward the variable resistor VR in order to charge the condenser C, and is applied to the collector of the transistor Tr via the stimulus plate 200.

By the way, the current flowing toward the variable resistor VR until the condenser C is charged up comes to flow toward the base of the transistor Tr in an amount proportional to the voltage drop between the resistors R1 and R2, so that the transistor Tr becomes in conduction state.

After the charging of the condenser C is finished, the condenser C acts as a decoupling condenser, so that the current is still applied to the base of the transistor Tr.

That is, the transistor Tr is turned on and thus the current can flow into the stimulus plate 200.

In FIG. 7, resistors R3 and R4 can be optionally included in the microcurrent generation unit 130 according to the value of the microcurrent.

According to another embodiment of the present invention, as shown in FIG. 8, the microcurrent generation unit 130 can generate microcurrent using the internal resistance r of the acupressure protrusions, which will be described below.

Accordingly, in order to generate several tens of microamperes of current, the internal resistance r of the acupressure protrusions 220 is required to meet the following condition.

The value of the internal resistance of the stimulus plate 200 is obtained from the following equation.

R=V/I (If 1 ampere of current flows when 1V of power is applied, a value of resistance is considered 1)

For example, when the supplied power is 3V, the acupressure protrusions 220 are designed in a manner such that the internal resistance is about 50 kΩ in order to cause 60 microamperes of current to flow. That is, microcurrent in a suitable range, 1 to 30 microamperes, can be supplied according to the value of the internal resistance of the acupressure protrusions 220.

Further, on the outer surface of the casing 110, the switch 140 for controlling the on/off operation of the microcurrent generation unit, and the indicator lamp 150 for indicating the remaining battery capacity are provided. The indicator lamp 150 may be an LED lamp, and may emit red, blue and yellow light rays according to the remaining battery capacity.

Still further, the switch 140 may have a flashing diode 152 which allows a user to identify whether the power of the battery 120 is supplied. Accordingly, the flashing diode 152 flashes at regular intervals while the switch 140 is in an on-state. The switch 140 continuously remains in an on-state while the microcurrent is supplied to body parts.

Next, the stimulus plate 200 will be described.

As shown in FIG. 5 and FIG. 6, the stimulus plate 200 has at least one acupressure patch 210 connected to the main body part 100 via an electric wire 230, and the acupressure patch 210 has the acupressure protrusions 220 which can stimulate meridian loci on the sole of a foot with microcurrent at a center portion thereof.

The stimulus plate 200 is made of a conductive material, and the acupressure protrusions 220 are sharp in order to enhance contact precision between the stimulus plate 200 and the body parts of a user.

The acupressure protrusions are preferably made of a specific magnetic metal.

That is, the acupressure protrusions 220 of the stimulus plate 200 are made of a conductive metal, such as silver or gold, and are covered with a metal thin film in order to increase the resistance thereof, so that microcurrent can flow.

The acupressure protrusions 220 of the stimulus plate 200 may be made of magnets, so that a magnetic field is formed on the stimulus plate 200.

In this case, complex physical energies, such as physical acupressure, conduction of microcurrent, and magnetic fields, can be applied to body parts.

Next, the coupling part 300 will be described below.

With reference to FIG. 5 and FIG. 6, the coupling part 300 according to the embodiment of the present invention is made of a cloth band having elasticity, and is designed to be worn around a user's ankle. The coupling part 300 serves to fix the main body part to a human body. The coupling part 300 is combined with the casing 110, and has a length adjustment unit 310 which allows a user to adjust the length of the coupling part 300.

Examples of the length adjustment unit 310 include a Velcro tape type (called ChickChickee), a buckle type, generally coupled to a waist band, and a ring type, generally applied to a bag. In the drawings, the ring type length adjustment unit 310 is disclosed.

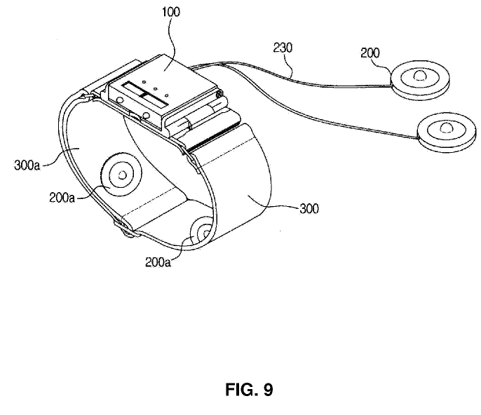

As shown in FIG. 9, according to the best embodiment of the present invention, at least one of the acupressure protrusions 220 of the stimulus plate 200 may be installed on the inner surface 300a of the coupling unit 300, so that meridian loci disposed at the ankle 2 can be stimulated with microcurrent. Accordingly, acupoints on the sole of a foot or the ankle are stimulated by complex physical energies of physical stimulation transferred through the stimulus plate 200, magnetic field, and electrical stimulation, so that blood flow is smoothed and the organs of the human body are positively affected.

The coupling part 300 according to another embodiment of the present invention may be a necklace type. If the coupling part 300 is a necklace type, a user can wear the main body part 100 like a pendant of a necklace using the coupling part 300. At this time, the stimulus plate 200, electrically connected to the main body part 100, is attached to meridian loci or meridian muscles at spots on the shoulders, neck, back or waist, which are suffering pains, so that those spots are stimulated with microcurrent.

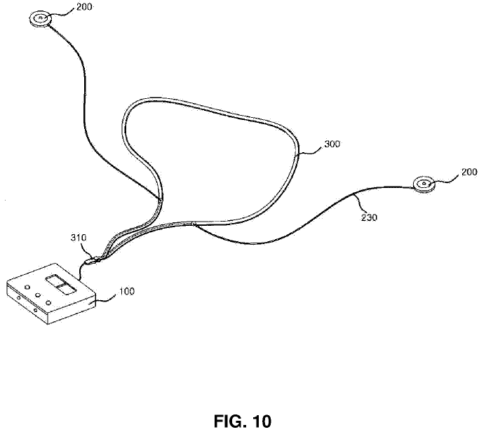

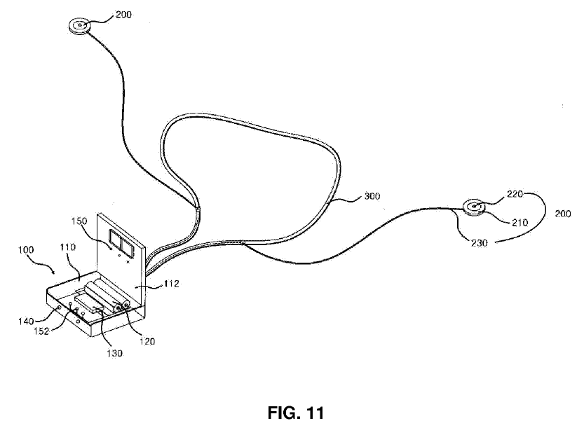

FIG. 10 is a perspective view illustrating the necklace type microcurrent stimulus apparatus according to another embodiment of the present invention, and FIG. 11 is a perspective view illustrating the internal structure of the necklace type microcurrent stimulus apparatus.

With reference to FIG. 10 and FIG. 11, the coupling part 300 according to another embodiment of the present invention has a necklace shape, an electric wire 230 is embedded in the ring-shaped coupling part 300, and the wire 230 is pulled out at a portion of the coupling part 300 and connected to the stimulus plate 200.

The necklace type coupling part 300 has a connector 320 which enables easy coupling and decoupling of the coupling unit 300 to and from the casing 110. Furthermore, the length of the necklace coupling unit 300 can be adjusted.

Hereinafter, the present invention will be described in more detail with reference to embodiments.

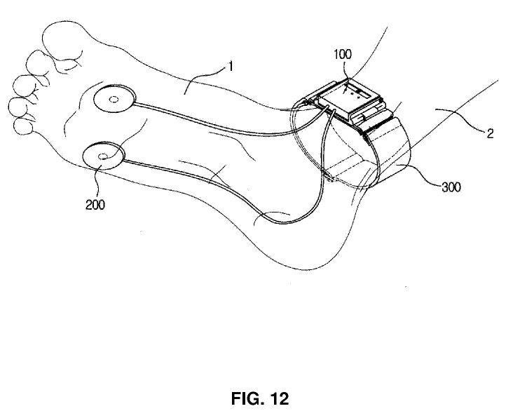

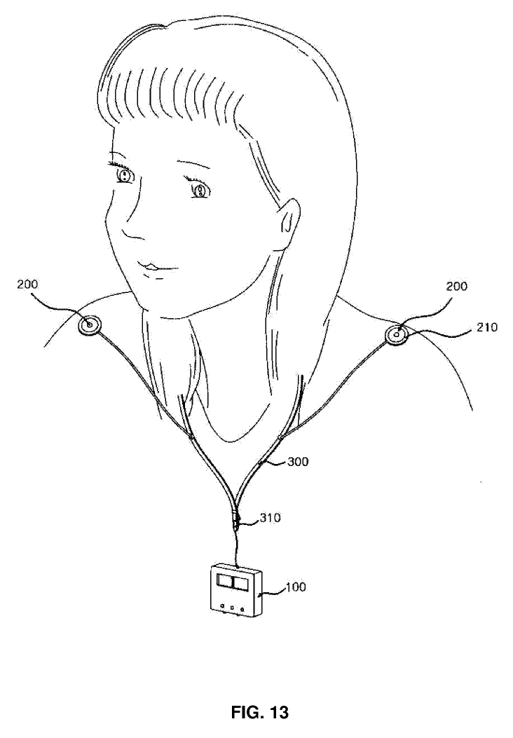

FIG. 12 illustrates the state of the microcurrent stimulus apparatus, according to the present invention, being worn.

Referring to FIG. 12, a user wears the main body part 100 around his or her ankle 1, and the main body part 100 is not separated from the user's body part while the user walks because it is fixed to the user's body part with the coupling part 300 combined with the casing 110 using the length adjustment unit 310.

The stimulus plate 200 connected to the main body 100 via a wire is installed between the sole of a foot and the sole of a shoe.

The stimulus plate 200 can be selectively attached to meridian loci on the sole of a foot by the acupressure patch 210.

In particular, it is desirable that the stimulus plate 200 be attached to YongcheonHyul, disposed at a concave portion of the big toe and the center portion of the front side of the sole of the foot.

The sole of the foot has a large number of important Hyuls (spots for meridian). Out of many Hyuls, the YongcheonHyul is considered highly important for use of the microcurrent stimulus apparatus because the YongcheonHyul is a meridian locus where wellness (vital energy) flows, and stimulation given to the YongcheonHyul activates the kidney, removes toxicity from water, smoothens the blood flow, and alleviates fatigue and languidness caused by the toxicity of water. That is, the YongcheonHyul is a meridian locus which is capable of easing fatigue in a very short time but is hidden in the concave portion of the sole of a foot, which is the most badly abused body part, so it is not stimulated during the normal daily life of a user.

According to another embodiment of the present invention, since the acupressure patch 210 of the stimulus plate 200 is attached to the inner surface 300a of the coupling unit 300, it is possible to stimulate the meridian loci at the ankle as well as those at the sole of a foot, and thus incretion is accelerated and fatigue is eased in a short time.

FIG. 13 is a view illustrating the state of the necklace type microcurrent stimulus apparatus, according to another embodiment of the present invention, being worn.

Referring to FIG. 13, it is desirable that a user use the apparatus by wearing the coupling unit 300 coupled to the main body part 100. Further, a user can hold the main body part 100 or put the main body part 10 on the floor when using the apparatus.

The stimulus plate 200, connected to the main body part 100 via the wire 230, is installed on a shoulder. The stimulus plate 200 can be attached to a body part, such as the shoulders, neck, back, or waist, using the acupressure patch 210.

Although the present invention has been described with reference to the embodiments described above, those skilled in the art will appreciate that various modifications, additions and substitutions are possible, without departing from the scope and spirit of the invention as disclosed in the accompanying claims.

### INDUSTRIAL APPLICABILITY

As described above, the microcurrent stimulus apparatus according to the present invention has advantageous effects in that it can stimulate body parts with microcurrent so that a user receives a massage effect at meridian loci and meridian muscles distributed all over the body parts while the user wears and carries it around his or her neck or ankle, and thus blood flood of blood vessels around stiff muscles and lesion spots becomes smooth, circulation of vital energy and blood can be normalized, and natural heating power is increased.

Further, the microcurrent stimulus apparatus according to the present invention can be widely used for treating obesity and pains.

What is claimed is:

1. A microcurrent stimulus apparatus, comprising:
a main body part including a casing, a battery installed in the casing, a microcurrent generation unit for converting power from the battery to microcurrent, a switch provided on an outer surface of the casing for controlling supply of the power supplied to the microcurrent generation unit from the battery, and an indicator lamp for indicating remaining battery capacity;
a stimulus plate having at least one acupressure patch electrically connected to the main body part and exposed outside the casing by protruding from an outer surface of the casing, in which acupressure protrusions, which can stimulate meridian loci or meridian muscles of body parts with microcurrent, are provided at a center portion of the acupressure patch; and
a coupling part which is combined with the main body part and allows a user to wear the main body part on a user's body part.

2. The microcurrent stimulus apparatus according to claim 1, wherein the microcurrent generation unit comprises a condenser C to which power from the battery is supplied, resistors R1 and R2, a transistor Tr1 and a variable resistor VR which are electrically connected to each other, wherein the microcurrent is generated in a manner such that if the switch is turned on, the power from the battery is supplied to the variable resistor VR in order to charge the condenser C, and is simultaneously supplied to a collector of the transistor Tr, current flowing toward the variable resistor VR until the condenser C is charged up flows to a base of the transistor Tr in an amount proportional to a voltage drop between the resistors R1 and R2, and then the condenser C serves as a decoupling condenser once the condenser C is charged up, thus the current still flows to the base of the transistor Tr.

3. The microcurrent stimulus apparatus according to claim 1, wherein the microcurrent generation unit generates the microcurrent using internal resistance of the acupressure protrusions.

4. The microcurrent stimulus apparatus according to claim 1, wherein the main body part includes a cover for allowing the battery to be replaced after the battery is used, and a flashing diode for allowing a user to identify whether the power is supplied, in which the flashing diode flashes at regular intervals while the switch is in an on state, and the switch is continuously in an on state when the microcurrent is generated.

5. The microcurrent stimulus apparatus according to claim 1, wherein the acupressure protrusions are made of magnets, so that the microcurrent stimulus apparatus further provides stimulation of a magnetic field.

6. The microcurrent stimulus apparatus according to claim 1, wherein at least one of the stimulus plates is installed on an inner surface of the coupling part, so the at least one of the stimulus plates can stimulate meridian loci at an ankle with the microcurrent.

7. The microcurrent stimulus apparatus according to claim 1, wherein the coupling part has a length adjustment unit for adjusting a length of the coupling part, and the length adjustment unit is a Velcro tape type, a buckle type, or a ring type.

8. The microcurrent stimulus apparatus according to claim 1, wherein the coupling part has a necklace shape so it can be worn by a user in a state in which the coupling part is combined with the main body part.
